(19) United States
(12) Patent Application Publication
(10) Pub. No.: US 2018/0263822 A1
(43) Pub. Date: Sep. 20, 2018

(54) Hearing Protection Device

(71) Applicant: Benjamin S. Rappaport, Short Hills, NJ (US)

(72) Inventor: Benjamin S. Rappaport, Short Hills, NJ (US)

(21) Appl. No.: 15/554,185

(22) PCT Filed: Feb. 26, 2016
(86) PCT No.: PCT/US16/19736

§ 371 Date: Aug. 28, 2017

Related U.S. Application Data
(60) Provisional application No. 62/176,730, filed on Feb. 27, 2015.

(51) Int. Cl. A61F 11/08

(52) U.S. Cl. CPC A61F 11/08; A61F 2011/085

(57) ABSTRACT

An exemplary hearing protection device includes a base portion, and an elongate mounting portion protruding from the base portion. The mounting portion includes a first end fixed to the base portion and a second end opposite the first end. The mounting portion decreases in thickness from the first end to a first point between the first end and the second end, increases in thickness from the first point to a second point between the first point and the second end, and decreases in thickness from the second point to the second end. The hearing protection device also includes a plug portion detachably secured to the mounting portion. The plug portion is configured to at least partially insert into a user's ear canal.

### CROSS-REFERENCE TO RELATED APPLICATION

This application claims the benefit of U.S. Provisional Application No. 62/176,730, filed Feb. 27, 2015.

### TECHNICAL FIELD

This disclosure relates to hearing protection devices.

### BACKGROUND

Disposable and non-disposable hearing protection devices soften, reduce or eliminate the amount of noise, dust, pollution, water, and wind that reaches a person's ear. Example hearing protection devices include ear plugs (e.g., devices that are inserted into a portion of person's ear) and ear muffs (e.g., devices that are placed over the exterior of a person's ear and shield the ear from noise, dust, pollution, water, and wind). Hearing protection devices are often used in noisy environments to protect a user's hearing in a broad range of different environments, settings, and/or purposes. For example, hearing protection devices are often used during motor cross and automotive racing events, sport shooting events, athletic events, parades, festivals, sporting events, and musical events. As another example, hearing protection devices are also often used in airports, construction sites, gun ranges, drilling sites, mining sites, hospitals (e.g., in MRI facilities), factories, laboratories, stadiums. As yet another example, hearing protection devices are often used in during relaxation activities (e.g., yoga and meditation) or as a sleep aid. As yet another example, hearing protection devices are often used during military operations (e.g., while firing ordinance, flying an aircraft, or driving a vehicle). As yet another example, hearing protection devices are often used during athletic activities (e.g., during swimming). As yet another example, hearing protection devices are often used during air travel (e.g., to reduce noise and/or the effects of changing cabin pressure). As yet another example, hearing protection devices are often used to reduce hearing loss, tinnitus, and other medical conditions. As yet another example, hearing protection devices are often used by those with special medical needs (e.g., autism).

### SUMMARY

In general, in an aspect, a hearing protection device includes a base portion, and an elongate mounting portion protruding from the base portion. The mounting portion includes a first end fixed to the base portion and a second end opposite the first end. The mounting portion decreases in thickness from the first end to a first point between the first end and the second end, increases in thickness from the first point to a second point between the first point and the second end, and decreases in thickness from the second point to the second end. The hearing protection device also includes a plug portion detachably secured to the mounting portion. The plug portion is configured to at least partially insert into a user's ear canal.

Implementations of this aspect may include or more of the following features.

In some implementations, the mounting portion can monotonically decrease in thickness from the first end to the first point, monotonically increase in thickness from the first point to the second point, and monotonically decrease in thickness from the second point to the second end.

In some implementations, the plug portion can define a channel, and the mounting portion can be configured to insert into the channel to secure the mounting portion of the plug portion. An inner diameter of the channel can vary between a first end of the channel and a second end of the channel. The channel can be defined by an elastic material. The channel can extend across a full length of the plug portion.

In some implementations, the mounting portion can be substantially continuous between the first end and the second end.

In some implementations, the plug portion can include a compressible material.

In some implementations, the base portion can include a decorative portion that extends outside of the user's ear when the plug portion is inserted into the user's ear canal.

In some implementations, the plug portion can be configured to attenuate sound entering into the user's ear when the plug portion is at least partially inserted into the user's ear canal.

In general, in another aspect, a hearing protection device includes a base portion, and an elongate mounting portion protruding from the base portion. The mounting portion includes a first end fixed to the base portion, a second end opposite the first end, and a protrusion between the first and second ends. The hearing protection device also includes a plug portion detachably secured to the mounting portion. The plug portion is configured to at least partially insert into a user's ear canal.

Implementations of this aspect may include or more of the following features.

In some implementations, the plug portion can define a channel, and the mounting portion can be configured to insert into the channel to secure the mounting portion of the plug portion.

In some implementations, the channel can define a recess for receiving the protrusion on the mounting portion.

In general, in another aspect, a hearing protection device includes a base portion, and an elongate mounting portion protruding from the base portion. The mounting portion includes a first end fixed to the base portion, a second end opposite the first end, and a first annular protrusion and a second annular protrusion disposed between first end and the second end. The hearing protection device also includes a plug portion detachably secured to the mounting portion and configured to at least partially insert into a user's ear canal. The plug portion defines a channel having a first recess configured to accept the first annular protrusion and a second recess configured to accept the second annular protrusion.

Implementations of this aspect may include or more of the following features.

In some implementations, the plug portion can include a flexible material, and wherein the channel is defined by the flexible material. The flexible material can include a rubber.

In some implementations, the plug portion can include a compressible material, and the compressible material can be disposed along an outer periphery of the flexible material. The compressible material can include a foam.

In general, in another aspect, a hearing protection device includes a base portion defining a channel, and a clamp portion having a plurality of clamp elements. The clamp element is at least partially disposed within the channel and the clamp element is configured to shift within the channel between an open position and a closed position. The hearing protection device also includes a plug portion detachably secured to the base portion via the clamp member and configured to at least partially insert into a user's ear canal. In the closed position, the plurality of clamp elements grasp a periphery of the plug portion to secure the plug portion to the base portion. In the open position, the plurality of clamp elements release the plug portion from the base portion.

Implementations of this aspect may include or more of the following features.

In some implementations, the clamp portion can include at least two clamp elements.

In some implementations, the clamp portion can further include a button, and a spring disposed between the clamp elements and the button. When the button is depressed, the spring can shift the clamp portion to the open position. When the button is released, the spring can shift the clamp portion to the closed position.

In general, in another aspect, a hearing protection device can include a base portion defining a first channel, and a mounting portion at least partially disposed within the first channel and configured to longitudinally shift within the first channel between a first position and a second position. The mounting portion includes an elongate shaft member, and a plurality of support elements. The hearing protection device also includes a plug portion detachably secured to the base portion via the support elements and configured to at least partially insert into a user's ear canal. The plug portion defines a second channel. In the first position, the shaft member radially biases the support elements against an interior of the second channel of the plug portion to secure the plug portion to the base portion. In the second position, the shaft member radially biases the support elements away from the second channel of the plug portion to release the plug portion from the base portion.

Implementations of this aspect may include or more of the following features.

In some implementations, the shaft member can include a groove configured to receive the support elements. In the first position, the support elements can be positioned within the groove. In the second position, the support elements can be positioned outside of the groove.

In some implementations, the groove can be defined along an outer circumference of the shaft member.

In some implementations, the mounting portion can further include a button, and a spring disposed about a periphery of the elongate shaft member. When the button is depressed, the mounting portion can shift to the open position. When the button is released, the mounting portion can shift to the closed position.

In general, in another aspect, a hearing protection device includes a base portion, wherein the base portion defines a first channel, and a mounting portion at least partially disposed within the first channel and configured to rotate within the channel between a first rotational position and a second rotational position. The mounting portion includes a gear member, and a plurality of support elements. The hearing protection device also includes a plug portion detachably secured to the base portion via the support elements and configured to at least partially insert into a user's ear canal. The plug portion defines a second channel. In the first rotational position, the gear member radially biases the support elements against an interior of the second channel of the plug portion to secure the plug portion to the base portion. In the second rotational position, the shaft member radially biases the support elements away from an interior of the second channel of the plug portion to release the plug portion from the base portion.

Implementations of this aspect may include or more of the following features.

In some implementations, the gear member can include a plurality of grooves each configured to receive one of the support elements. In the first rotational position, the support elements can be positioned within a respective groove. In the second rotational position, the support elements can be positioned outside of a respective groove. The grooves can be angularly spaced about a circumference of the gear member.

In some implementations, the first rotational position and the second rotational position can differ by approximately 30°.

In general, in another aspect, a hearing protection device includes a base portion including a first magnet, and a plug portion including a second magnet. The plug portion is detachably secured to the mounting portion via a magnetic force between the first magnetic and the second magnet. The plug portion is configured to at least partially insert into a user's ear canal.

In some implementations, the first magnet can be disposed within a first recess defined by the base portion, and the second magnet can be disposed within a second recess defined by the plug portion.

In general, in another aspect, a hearing protection device includes a base portion, and an elongate mounting portion protruding longitudinally from the base portion. The mounting portion includes a plurality of first teeth protruding radially from the mounting portion. The hearing protection device also includes a plug portion defining a longitudinally extending channel. The plug portion includes a plurality of second teeth protruding radially into the channel, and wherein the plug portion is detachably secured to the base portion via the first teeth and second teeth. The plug portion is configured to at least partially insert into a user's ear canal. When the elongate mounting portion is in a first rotational position relative to the base portion, the first teeth engage with the second teeth to secure the plug portion to the base portion. When the elongate mounting portion is in a second rotational position relative to the base portion, the first teeth disengage from the second teeth to release the plug portion from the base portion.

Implementations of this aspect may include or more of the following features.

In some implementations, one or more of the first teeth can protrude radially from the mounting portion in a first direction. One or more of the first teeth can protrude radially from the mounting portion in a second direction opposite the first direction.

In some implementations, one or more of the second teeth can protrude radially into the channel in a first direction. One or more of the second teeth can protrude radially into the channel in a second direction opposite the first direction.

In some implementations, the first rotational position and the second rotational position can differ by approximately 90°.

In some implementations, the first teeth can be angularly spaced about a circumference of the mounting portion.

In some implementations, the second teeth can be angularly spaced about a circumference of the channel.

In some implementations, the first rotational position and the second rotational position can differ by approximately 45°.

In some implementations, the first teeth and the second teeth can be configured such that when the elongate mounting portion is in a first longitudinal position relative to the base portion, elongate mounting portion and base portion are rotationally locked. Further, when the elongate mounting portion is in a second longitudinal position relative to the base portion, elongate mounting portion and base portion are not rotationally locked.

In general, in another aspect, a hearing protection device includes a base portion, and a mounting portion including a first arm and a second arm, and configured to form first and second positions. The first arm includes a first protrusion proximate to an end of the first arm, and a first support element proximate to another end of the first arm. The second arm includes a second protrusion proximate to an end of the second arm, and a second support element proximate to another end of the second arm. The first arm and the second arm are coupled to each other via a hinge. The first arm and the second arm are configured to move with respect to each other via the hinge. The hearing protection device also includes a plug portion detachably secured to the base portion via the support elements and configured to at least partially insert into a user's ear canal. The plug portion defines a channel. In the first position, the mounting portion biases the first support element and the second support elements against an interior of the channel of the plug portion to secure the plug portion to the base portion. In the second position, the mounting portion biases the first support element and the second support elements away from the interior of the channel of the plug portion to release the plug portion to the base portion.

Implementations of this aspect may include or more of the following features.

In some implementations, the first protrusion and the second protrusion can protrude from the base portion. When the first protrusion and the second protrusion are depressed, the mounting portion can be placed into the first position. When the first protrusion and the second protrusion are released, the mounting portion can be placed into the second position; and

In some implementations, the plug portion can further define an annular groove within the channel. In the first position, the mounting portion can shift the first support element and the second support element into the groove. In the second position, the mounting portion can shift the first support element and the second support element out of the groove.

In some implementations, the mounting portion can further include a spring coupled to the first arm and the second arm. When the first protrusion and the second protrusion are released, the mounting portion can be placed into the second position via a spring force against the first arm and the second arm.

In general, in another aspect, a hearing protection device includes a base portion, and an elongate mounting portion protruding from the base portion. The elongate mounting portion includes an outer sleeve defining a plurality of apertures, a plurality of moveable support elements protruding from the apertures, and a spring element disposed between the support elements. The hearing protection device also includes a plug portion detachably secured to the base portion via the support elements and configured to at least partially insert into a user's ear canal. The plug portion defines a channel. The elongate mounting portion is configured such that when the elongate mounting portion is inserted a first distance into the channel, the support elements into the apertures, and when the elongate mounting portion is inserted a second distance into the channel greater than the first distance, the support elements protrude from the apertures into a recess defined within the channel.

Implementations of this aspect may include one or more of the following features.

In some implementations, the elongate mounting portion can be configured such that when the elongate mounting portion is withdrawn from within the channel by a third distance, the support elements retract into the apertures, when the elongate mounting portion is withdrawn from within the channel by a fourth distance greater than the third distance, the support elements protrude from the apertures.

In some implementations, the elongate mounting portion can further include an inner sleeve disposed within the outer sleeve and surrounding the spring element.

The details of one or more embodiments are set forth in the accompanying drawings and the description below. Other features and advantages will be apparent from the description and drawings, and from the claims.

### DESCRIPTION OF DRAWINGS

FIGS. 1A-C are diagrams of an exemplary hearing protection device.

FIG. 2 is a diagram of an exemplary hearing protection device inserted into a user's ear.

FIG. 3 is a diagram of a hearing protection device having an exemplary mechanism for coupling a base portion of the hearing protection device to a plug portion of the hearing protection device.

FIG. 4 is a diagram of a hearing protection device having another exemplary mechanism for coupling a base portion of the hearing protection device to a plug portion of the hearing protection device.

FIG. 5A and 5B are diagrams of a hearing protection device having another exemplary mechanism for coupling a base portion of the hearing protection device to a plug portion of the hearing protection device.

FIG. 6 is a diagram of a hearing protection device having another exemplary mechanism for coupling a base portion of the hearing protection device to a plug portion of the hearing protection device.

FIG. 7A is a diagram of a hearing protection device having another exemplary mechanism for coupling a base portion of the hearing protection device to a plug portion of the hearing protection device.

FIGS. 7B and 7C are diagrams of the exemplary mechanism shown in FIG. 7A.

FIG. 8 is a diagram of a hearing protection device having another exemplary mechanism for coupling a base portion of the hearing protection device to a plug portion of the hearing protection device.

FIG. 9A is a diagram of another exemplary mechanism for coupling a base portion of the hearing protection device to a plug portion of the hearing protection device.

FIGS. 9B and 9C are diagrams of a hearing protection device including the exemplary mechanism shown in FIG. 9A.

FIG. 10A is a diagram of a hearing protection device having another exemplary mechanism for coupling a base portion of the hearing protection device to a plug portion of the hearing protection device.

FIGS. 10B-D are diagrams of the exemplary mechanism shown in FIG. 10A.

FIGS. 11A and 11B are diagrams of a hearing protection device having another exemplary mechanism for coupling a base portion of the hearing protection device to a plug portion of the hearing protection device.

FIG. 12 is a diagram of a hearing protection device having another exemplary mechanism for coupling a base portion of the hearing protection device to a plug portion of the hearing protection device.

### DETAILED DESCRIPTION

Various implementations of a hearing protection device are described herein. One or more of these implementations can be used to protect a user's hearing. For example, a user can position all or part of a hearing protection device into a portion of his ear (e.g., his ear canal). Once positioned, the hearing protection device can reduce or eliminate the amount of sound that reaches the person's ear. This can be useful, for example, to protect the user's hearing in noisy environments, such as automotive races, gun ranges, airports, and construction sites.

In some implementations, a hearing protection device can include multiple different portions. For example, a hearing protection device can include a plug portion that is configured to insert into a user's ear (e.g., into a user's ear canal), and a base portion that is configured to remain outside of a user's ear during use. This can be beneficial, for example, as it allows a portion of the device (e.g., a plug portion) to be inserted into a person's ear to reduce or eliminate noise entering the ear, while allowing another portion of the device (e.g., the base portion) to remain outside of a person's ear such that the device can be physically grasped by a user and removed from the ear after use. This configuration can also provide aesthetic benefits. For example, the portion of the device that remains outside of the ear can include a decorative or aesthetically pleasing design which can be viewed by others during use of the device.

In some cases, these portions can be reversibly coupled to each other, such that a user can separate or detach the base portion from the plug portion, and subsequently reattach the base portion to the plug portion. This can be beneficial, for example, as it allows the user to selectively remove, replace, and/or customize specific portions of the hearing protection device, while keeping the other portions of the hearing protection device intact. For example, a base portion can be replaced to alter the appearance of the device during use, while keeping the same plug portion so that the fit of the device within the user's ear canal remains unchanged. As another example, a plug portion can be replaced to change the fit of the device within the user's ear canal (e.g., by replacing the plug portion with another plug portion having a different size and/or shape), while keeping the same base portion so that the appearance of the device remains the same during use.

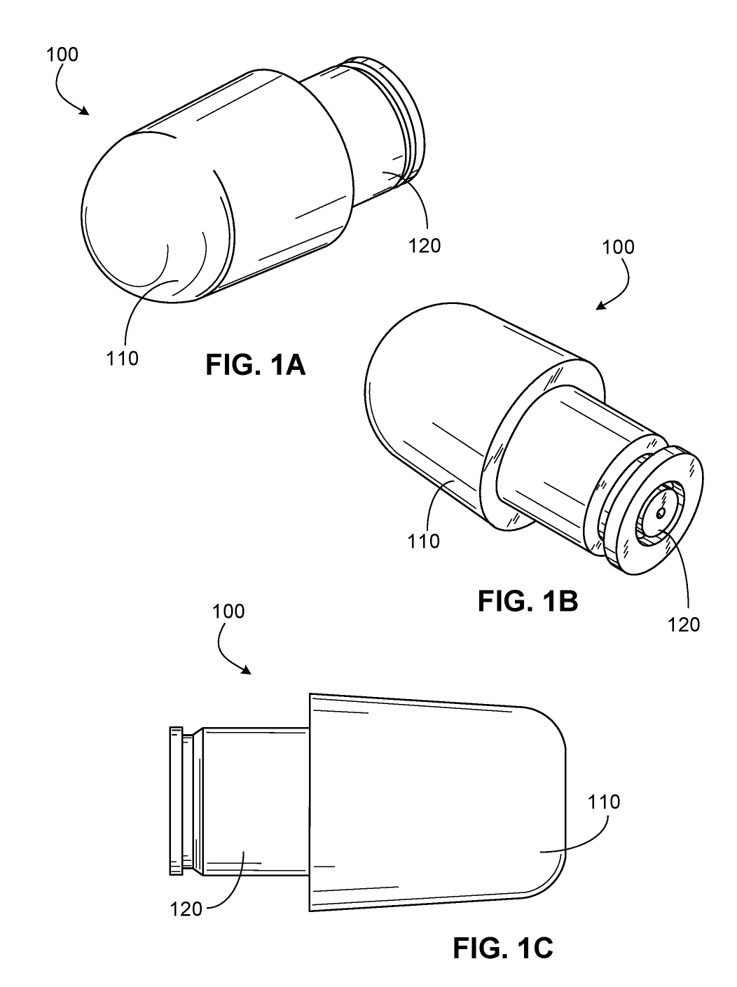

An exemplary implementation of a hearing protection device 100 is shown in FIG. 1A (depicting a front perspective view of the hearing protection device 100), FIG. 1B (depicting a rear perspective view of the hearing protection device 100), and FIG. 1C (depicting a side view of the hearing protection device 100). The hearing protection device 100 includes a plug portion 110 and a base portion 120.

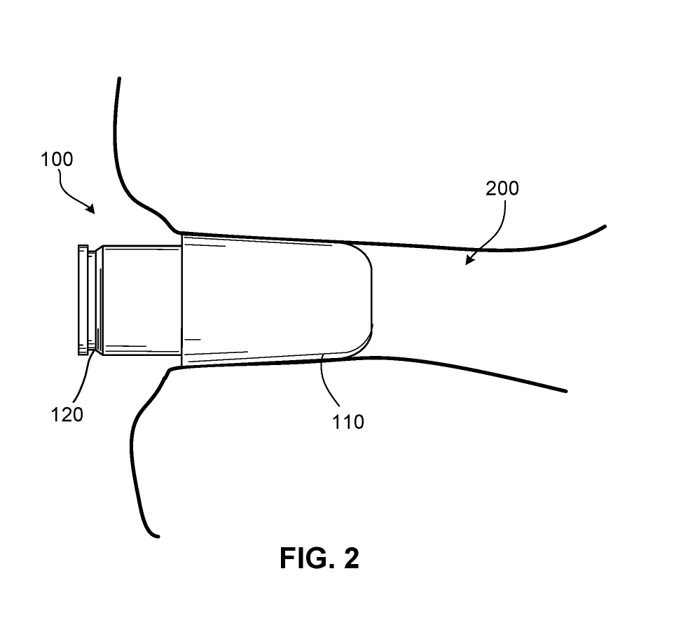

The plug portion 110 has a generally frusto-conical shape, and is configured to insert into a user's ear. For example, as shown in FIG. 2, the plug protection device 100 can be positioned within a user's ear canal 200 to physically block the ear canal 200. Thus, sound entering the ear canal 200 is attenuated or eliminated by the plug portion 110. Further, the plug portion 110 can also reduce or eliminate dust, pollution, water, and/or wind from entering the ear canal 200.

The plug portion 110 can be constructed using a compressible, flexible, expandable, and/or elastic material. Example of suitable materials include wax, foam, memory foam, vinyl, polyvinyl chloride, polyurethane, acrylic, rubber, plastic, and silicone. A compressible and/or flexible material can be beneficial, for example, as it allows the plug portion 110 to bend or compress to conform to the physical dimensions of the ear canal 200. Thus, the plug portion 110 can fit more closely and more securely within the ear canal 200. In some cases, the plug portion 110 can be constructed such that its physical dimensions are larger than the ear canal 200, but such it can still be compressed to fit within the ear canal 200. Once placed in the ear canal 200, the plug portion 110 expands to against the ear canal 200. This can be beneficial, for example, as it further secures the plug portion 110 within the ear canal 200.

In some cases, the plug portion 110 can include a filter that acts as a pressure regulator. For example, the plug portion 110 can include a porous ceramic filter. One end of the ceramic filter can be exposed to the ambient pressure, and the opposite end can be exposed to the interior of a user's ear. As the ambient pressure changes, a pressure differential is created across the ceramic filter, causing air to flow through the porous filter. The flow of air is impeded by the filter. Thus, the effects of rapid changes in ambient pressure (e.g., as experienced during air travel) are lessened.

Although an exemplary plug portion 110 is shown in FIGS. 1A-C and FIG. 2, this is merely an illustrative example. In practice, the size and/or shape of the pug portion 110 can vary, depending on the implementation. For example, instead of a frusto-conical shape, the plug portion 110 can have some other shape (e.g., a cylindrical shape, a polygonal shape, a conical shape, an arbitrary shape, or some combination thereof).

As another example, in some cases, the plug portion 110 can be configured such that an entirety of the plug portion 110 can be inserted into a person's ear. For example, the plug portion 110 can be sized and shaped such that it can be fully inserted into the ear canal 200. In some cases, the plug portion 110 can be configured such that only a portion of the plug portion 110 can be inserted into a person's ear. For example, the plug portion 110 can be sized and shaped such that it cannot be fully inserted into the ear canal 200 (e.g., having a length and/or width that is too large to allow the plug portion 110 to fully insert into the ear canal 200).

The base portion 120 has a generally cylindrical shape, and is configured to remain, at least in part, outside of the ear canal 200 during use. For example, as shown in FIG. 2, when the hearing protection device 100 is positioned within a user's ear canal 200, the base portion 120 can remain outside of the ear canal 200. This can be useful, for example, as it allows the user to physically grasp the base portion 120, such that the hearing protection device 100 can be removed after use. This also allows the user to physically grasp the base portion 120 to insert the hearing protection device 100 into the ear canal 130, and to adjust its placement once inserted.

As the base portion 120 remains, at least in part, outside of the ear canal 200 during use, the base portion 120 can be configured to have a particular decorative appearance. This can be beneficial, for example, as it allow the user to use the hearing protection device as a piece of decoration, jewelry, or fashion. As an example, as shown in FIGS. 1A-C and FIG. 2, the base portion 120 has the physical appearance of a bullet. Thus, when the hearing protection device 100 is inserted into the ear canal 200, it appears to be a bullet lodged into the person's ear.

The base portion 120 can be constructed using of a relatively rigid material. Example materials include plastic, metal, wood, ceramic, stone, crystal, carbon fiber, rubber, rubber or fiber composite materials, glass, and other relatively rigid materials. A rigid material can be beneficial, for example, as it allows the user to grasp the base portion 120 more securely. However, in some cases, the base portion 120 can be constructed using a compressible and/or flexible material, either instead of or in addition to a rigid material.

Although an exemplary base portion 120 is shown in FIGS. 1A-C and FIG. 2, this is also merely an illustrative example. In practice, the size and/or shape of the base portion 120 can also vary, depending on the implementation. For example, the base portion 120 can have a cylindrical shape, a polygonal shape, a conical shape, an arbitrary shape, or some combination thereof.

Further, although the base portion 120 is shown as having the physical appearance of a bullet, in the practice, the physical appearance can also vary. For example, in some cases, the base portion 120 can have a physical appearance of another object, such as a car, a car tire, a flower, a butterfly, or any other object. In some cases, the base portion 120 can have a particular color scheme or visual pattern. For example, the base portion 120 can be painted such that it portrays particular images, text, and/or patterns on its exterior.

As another example, in some cases, the base portion 120 can be configured such that it is longer, shorter, wider, and/or thinner than shown in FIGS. 1A-C and FIG. 2. Further, in some cases, the base portion 120 can be configured such that it differs in size relative to the plug portion 110. For example, as shown in FIGS. 1A-C and FIG. 2, the base portion 120 has a smaller diameter than the plug portion 110; however, in some cases, the base portion can have the same diameter or a larger diameter than the plug portion 110.

In some cases, the plug portion 110 and the base portion 120 can be reversibly coupled to each other, such that a user can separate or detach the base portion 120 from the plug portion 110, and subsequently reattach the base portion 120 to the plug portion 110. This can be beneficial, for example, as it allows the user to selectively remove, replace, and/or customize specific portions of the hearing protection device, while keeping the other portions of the hearing protection device intact.

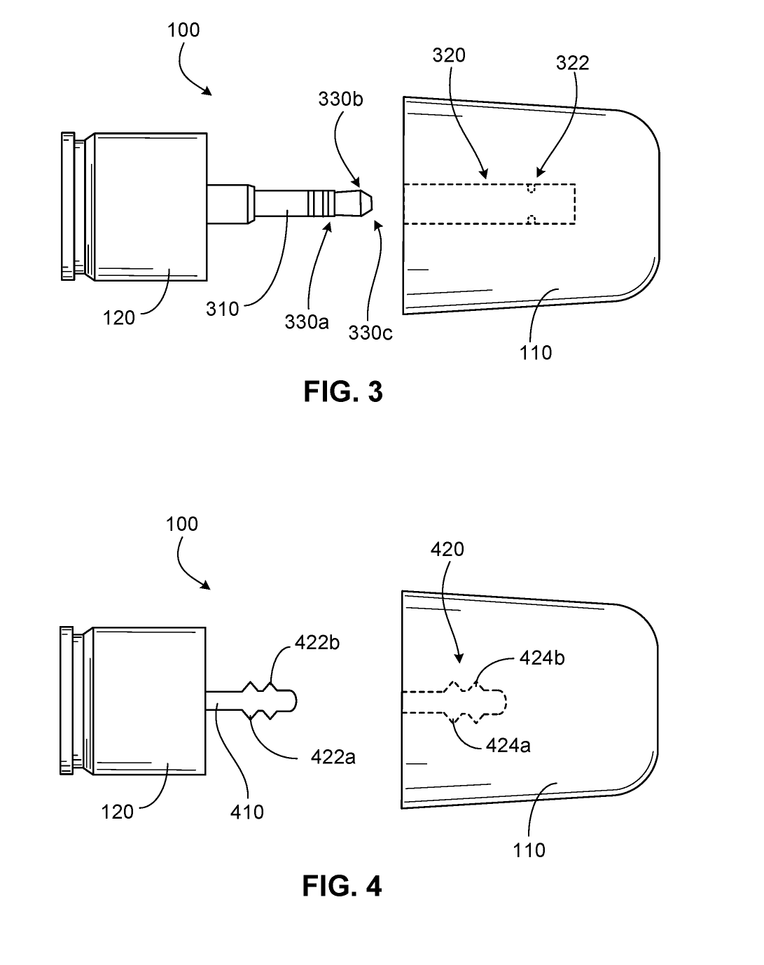

This reversible coupling feature can be provided in various ways. As an example, as shown in FIG. 3, a plug portion 110 can be coupled to a base portion 120 through an elongate mounting portion 310. The mounting portion 310 is attached to and protrudes from the base portion 120, and is configured to insert into a corresponding channel 320 defined within the plug portion 110. When the mounting portion 310 is inserted into the channel 320, the plug portion 110 and the base portion 120 are physically secured to each other. Further, the plug portion 110 and the base portion 120 can be physically separated from each other by pulling the plug portion 110 and the base portion 120 away from one another.

The mounting portion 310 can be generally elongate or shaft-like in shape. In some cases, the mounting portion 310 can have a circular cross-section. In other cases, the mounting portion 310 can have other cross-sections, such as a polygonal, ovular, or any other shape.

In some cases, the mounting portion 310 can include variations in thickness (e.g., variations in its diameter) that allow the mounting portion 310 to fit more securely within the channel 320. For example, as shown in FIG. 3, the mounting portion 310 can initially decrease (e.g., monotonically) in thickness in the direction from the base portion 120 to a point 330a, then increase (e.g., monotonically) in thickness in the direction from the point 330a to a point 330b, then decrease (e.g., monotonically) in thickness in the direction from the point 330b to the end point 330c of the mounting portion 310. This arrangement provides the mounting portion 310 with an annular recess about the point 330a. Further, the channel 320 includes an annular ridge 322 along a wall of the channel 320. The ridge 322 is positioned within the channel 320 such that when the mounting portion 310 is inserted into the channel 320, the ridge 322 aligns with the annular recess about the point 330a, thereby securing the mounting portion 310 in place.

The channel 320 can be generally tube-like in shape. In some cases, the channel 320 can have a circular cross-section, or some other cross-sectional shape similar to that of the mounting portion 310. In some cases, the channel 320 can be defined by an elastic material, such as rubber, elastic foam, silicone, or elastic plastic. This allows the walls of the channel 320 to bend outward to accept the mounting portion 310 during insertion (e.g., as the point 330b passes across the ridge 322), and to allow the mounting portion 310 to be removed (e.g., as the point 330b passes in the opposite direction across the ridge 322). In some cases, the diameter of the channel 320 can be slightly smaller than the diameter of the mounting portion 310, such that the channel 320 bends slightly outward when the mounting portion 310 is inserted into it. This can be beneficial, for example, as it further secures the mounting portion 310 within the channel 320 (e.g., through friction).

In some cases, the channel 320 can be defined by an elastic sleeve or spine that is embedded within the plug portion 110. In some cases, the elastic sleeve or spine can be constructed from the same material as the plug portion 110. In other cases, the elastic sleeve or spine can be constructed from a different material than the plug portion 110. For example, in some cases, the sleeve or spine can be construed from an elastic or flexible material (e.g., rubber), and can be surrounding by a compressible material (e.g., foam).

Although exemplary mounting portion 310 and corresponding channel 320 are shown in FIG. 3, this is merely an illustrative example. In practice, the mounting portion 310 and the channel 320 can differ, depending on the implementation. For example, in some cases, the mounting portion 310 can increase and/or decrease in thickness any number of times in succession in the direction from the base portion 120.

As another example, as shown in FIG. 4, a plug portion 110 can be coupled to a base portion 120 through an elongate mounting portion 410. In general, the plug portion 110, the base portion 120, and the mounting portion 410 can be similar to that shown in FIG. 3. For example, the mounting portion 410 is attached to and protrudes from the base portion 120, and is configured to insert into a corresponding channel 420 defined within the plug portion 110. When the mounting portion 410 is inserted into the channel 420, the plug portion 110 and the base portion 120 are physically secured to each other. Further, the plug portion 110 and the base portion 120 can be physically separated from each other by pulling the plug portion 110 and the base portion 120 away from one another.

In this example, however, the mounting portion 410 includes two annular protrusions 422a and 422b along its length. Further, the channel 420 includes two annular recesses 424a and 424b along a wall of the channel 420. The recess 424a and 424b are positioned within the channel 420 such that when the mounting portion 410 is inserted into the channel 420, the recesses 424a and 242b align with the annular protrusions 422a and 422b, respectively, thereby securing the mounting portion 410 in place.

Although two protrusions and two recesses are shown, this is merely an illustrative example. In practice, any number of protrusions and recesses can be used, depending on the implementation. For example, in some cases, a mounting portion can include one, two, three, four, or more protrusions, and the plug portion 110 can include one, two, three, four, or more corresponding recesses in a channel.

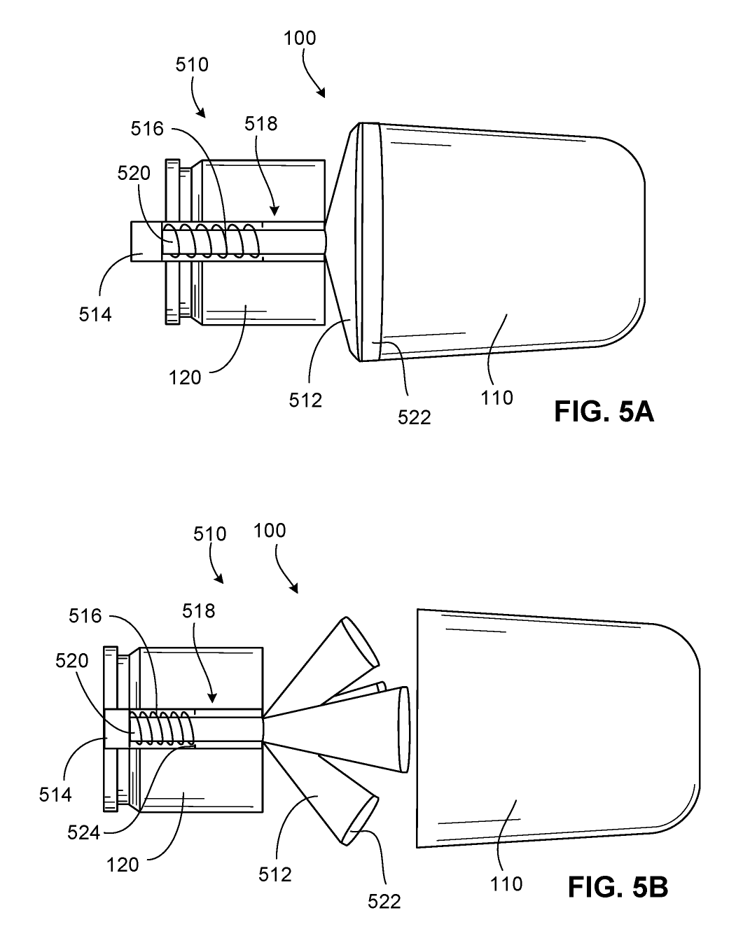

As another example, as shown in FIG. 5A, a plug portion 110 can be coupled to a base portion 120 through a clamp mechanism 510. The clamp mechanism 510 is disposed, at least in part, with the base portion 120. The clamp mechanism 510 includes several clamp elements 512, a button 514, and a spring 516 disposed about the proximity of the clamp elements 512 and proximate to the button 514. The clamp elements 512, the button 514 and the spring 516 can be disposed, at least in part, within a channel 518 defined within the base portion 120.

The clamp elements 512 are constructed from a flexible shape-memory material (e.g., alloys of zinc, copper, gold and/or iron) and have a “memory” corresponding to a relatively open or fanned out shape. As a result, the clamp elements 512 can be longitudinally shifted further into the channel 518 to grip the plug portion 120, and shifted further out of the channel 518 is release the plug portion 120. In some cases, the clamp elements 512 can collectively have a substantially cylindrical cross section within the channel 518, and a fanned out arrangement outside of the channel 518.

To illustrate, as shown in FIG. 5A, when the clamp mechanism 510 is in a closed position, the ends 520 of the clamp elements 512 are positioned relatively deep within the channel 518 (e.g., near the button 514). As described above, the clamp elements 512 have a “memory” corresponding to a relatively open or fanned out shape, and thus, will be physically biased towards an open or fanned out arrangement. However, due to the spring force of the spring 516, the clamp elements 512 are biased in the direction of the button 514 (i.e., in the left direction of FIG. 5A). As a result, a relatively large portion of each clamp element 512 is constrained within the channel 518, causing the gripping edges 522 of clamp elements 512 to be tightly clamped against the plug portion 110, and thereby securing the plug portion 110 to the base portion 120. Further, the spring 516 biases the button 514 outward from the base portion 120, such that it protrudes from the base portion 120.

As shown in FIG. 5B, the clamp mechanism 510 can be shifted to an open position to remove the plug portion 110 from the clamp elements 512. This can be performed, for example, by a user manually pressing the button 514 such that it moves further into the plug portion 110. The movement of the button 514 compresses the spring 516 against a spring wall 524, and forces or shifts the clamp elements 512 further out of the channel 518 from the opposite end (i.e., in the right direction of FIG. 5B). As a result, the clamp elements 512 are no longer as physically restricted within the channel 518. Because of their shape memory, the clamp elements 512 can open further outward, thereby releasing the plug portion 110 from the gripping edges 522.

The clamp mechanism 510 can be returned to the closed position by releasing the button 514. The spring 516 again biases the button 514 outward from the base portion 120, and allows the end 520 of the clamp elements 512 to shift further into the channel 518 (i.e., in the left direction of FIG. 5A). As a result, the clamp elements 512 are again shifted further into the channel 518, and the clamp elements 512 again clamp inward (e.g., against the plug portion 110).

Although four clamp elements 512 are shown, in practice, any number of clamp elements 512 can be used. For example, in some cases, the clamp mechanism 510 can include two, three, four, five, or more clamp elements 512. Further, although shape-memory metals are described above, in some cases, the clamp elements 512 can be made from other materials, such as aluminum, steel, iron, plastic, sterling silver, silver, titanium, and/or other materials.

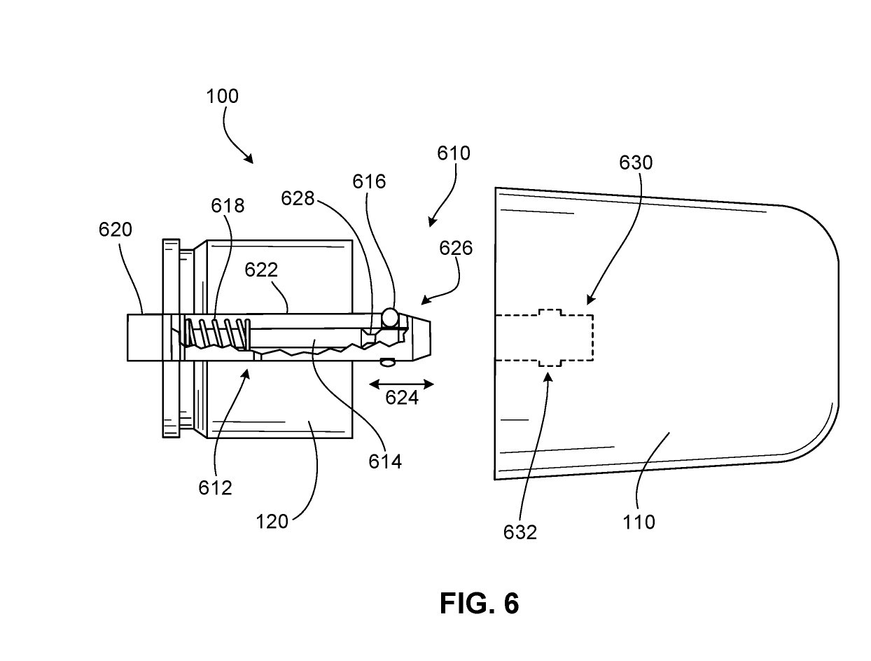

As another example, as shown in FIG. 6, a plug portion 110 can be coupled to a base portion 120 through a mounting mechanism 610. The mounting mechanism 610 is disposed within a channel 612 defined within the base portion 120, and includes an elongate shaft member 614 and several support elements 616 positioned along a periphery of the shaft member 614. The mounting mechanism 610 also includes a spring 618 positioned along a periphery of the shaft member 614, a button 620 that protrudes from base portion 120, and an outer sleeve 622 that houses the spring 618, the shaft member 614, and the support elements 616.

The shaft member 614 is configured to longitudinally shift within the channel 612 (e.g., as indicated by the double-headed arrow 624). However, the spring 618 biases the shaft member 614 inward into the base portion 120 (e.g., in the direction of the button 620). As shown in FIG. 6, in this position, the support elements 616 as disposed along an outer periphery of the shaft member 614, and thus are radially biased by the shaft member 614 such that they protrude from apertures 626 of outer sleeve 622 of the mounting mechanism 610.

However, when the button 620 is pressed inward into the base portion 120, the force upon the button 620 compresses the spring 618, causing the shaft member 614 to move outward from the base portion 120 (e.g., away from the button 620). This movement aligns an annular recess or groove 628 on an outer circumstance of the shaft member 614 with the support elements 616. As a result, the support elements recess into the annular recess or groove 628 and retract from the apertures 626 of the outer sleeve 622. Thus, when the button 620 is pressed, the support members 616 no longer protrude from the outer sleeve 622.

The mounting mechanism 610 can be used to securely and reversibly couple the base portion 120 to the plug portion 110. For example, to mount the base portion 120 to the plug portion 110, a user can press the button 620, causing the support elements 616 to retract. Thus, the outer sleeve 622 can be freely inserted into a corresponding channel 630 defined within the plug portion 110. When the button 620 is released, the support elements 616 are radially biased by the shaft member 614, and thus protrude from the outer sleeve 622. These support elements 616 are retained by corresponding recesses 632 defined within the channel 630 and protrude against the interior of channel 640. Thus, the protruding support elements 618 secure the mounting mechanism 610 in place within the channel 630, thereby coupling the base portion 120 to the plug portion 110. To release the base portion 120 from the plug portion 110, a user can again press the button 620—which causes the support elements 616 to be radially biased away from the channel and retract—and pull the base portion 120 away from the plug portion 110.

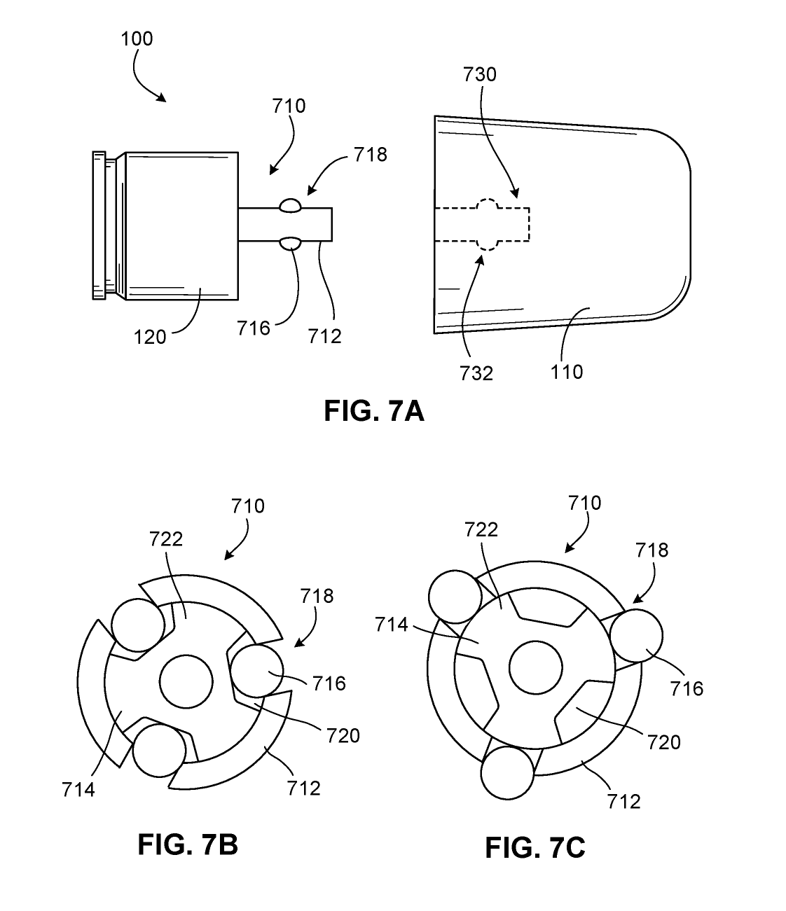

As another example, as shown in FIG. 7A, a plug portion 110 can be coupled to a base portion 120 through a mounting mechanism 710. The mounting mechanisms 710 is shown in cross-section in FIGS. 7B and 7C.

The mounting mechanism 710 is coupled to the base portion 120, and includes an outer sleeve 712, a gear 714 disposed within the outer sleeve 712, and several support elements 716 that protrude from apertures 718 of the outer sleeve 712.

The gear 714 is configured to rotate with respect to the outer sleeve 712 and the support elements 716. The rotational position of the gear 714 can be selected by a user. For example, the gear 714 can be rotationally locked to the base portion 120, such that when the base portion 120 is rotated with respect to the outer sleeve 712, the gear 714 rotates with the base portion 120.

As shown in FIG. 7B, when the gear 714 is in a first rotational position relative to the outer sleeve 712, the support elements 716 are aligned with recesses or grooves 720 of the gear 714. Thus, the support elements 716 retract from the outer sleeve 712.

As shown in FIG. 7C, when the gear 714 is in a second rotational position relative to the outer sleeve 712, the support elements 716 are positioned along the teeth 722 of the gear 714. Thus, the support elements 716 protrude from the apertures 718 of the outer sleeve 712.

The mounting mechanism 710 can be used to securely and reversibly couple the base portion 120 to the plug portion 110. For example, to mount the base portion 120 to the plug portion 110, a user can rotate the gear 714 relative to the outer sleeve 712 (e.g., by rotating the base portion 120) such that the gear 714 is in a first rotational position relative to the outer sleeve 712 (e.g., as shown in FIG. 7B). This causes the support elements 716 to retract. Thus, the outer sleeve 712 can be freely inserted into a corresponding channel 730 defined within the plug portion 110. To secure the plug portion 110 to the base portion 120, the user rotates the gear 714 relative to the outer sleeve 712 (e.g., by rotating the base portion 120) such that the gear 714 is in a second rotational position relative to the outer sleeve 712 (e.g., as shown in FIG. 7C). This causes the support elements 716 to be radially biased by the gear 714, and thus protrude from the outer sleeve 712. These support elements 716 are retained by corresponding recesses 732 defined within the channel 730 and protrude against the interior of channel 730. Thus, the protruding support elements 716 secure the mounting mechanism 710 in place within the channel 730, thereby coupling the base portion 120 to the plug portion 110.

To release the base portion 120 from the plug portion 110, a user can again rotate the gear 714 relative to the outer sleeve 712 (e.g., by rotating the base portion 120) such that the gear 714 is in the first rotational position relative to the outer sleeve 712 (e.g., as shown in FIG. 7C). This causes the support elements 618 to be radially biased away from the channel and retract. The user can then pull the base portion 120 away from the plug portion 110.

Three support elements 716 are shown in FIGS. 7B and 7C, each of which are equally distributed about a periphery of the gear 714. Thus, in this example, the first rotational position differs from the second rotation position by approximately 60°. However, in practice, any number of support elements 716 can be used (e.g., one, two, three, four, or more). Further, although the support elements 716 are shown as being equally distributed about a periphery of a gear 714, in practice, support elements 716 can be distributed in different patterns, such that the user can rotate the gear 714 a greater amount (e.g., more than 60°) or a lesser amount (e.g., less than 60°) to retract and/or deploy the support elements 716 from the outer sleeve 712.

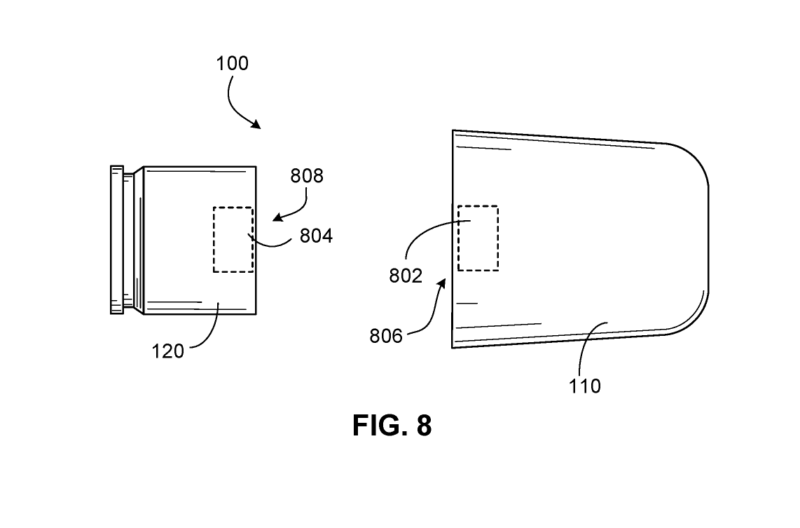

As another example, as shown in FIG. 8, a plug portion 110 can be coupled to a base portion 120 through magnets 802 and 804 mounted on or within the plug portion 110 and the base portion 120, respectively. For example, the magnet 802 can be positioned such that its outer face has one polarity (e.g., “north”), while the magnet 804 can be positioned such that its outer face has the opposite polarity (e.g., “south”). Thus, when the plug portion 110 and base portion 120 are positioned against each other, the outer faces of the magnets 802 and 804 attract one another and physically couple the plug portion 110 to the base portion 120.

In some cases, the magnets 802 and 804 can be mounted within recesses 806 and 808 defined in the plug portion 110 and base portion 120, respectively. In some cases, the magnets 802 and 804 can be mounted to a periphery of the plug portion 110 or base portion 120.

Although two magnets are shown in FIG. 8, in practice, any number of magnets can be used (e.g., two, three, four, five, or more magnets). Further, the position of the magnets can vary from that shown in FIG. 8. For example, instead of positioning magnets to a cross-sectional center of the plug portion 110 and/or base portion 120, magnets can be positioned along a periphery of the plug portion 110 and/or base portion 120.

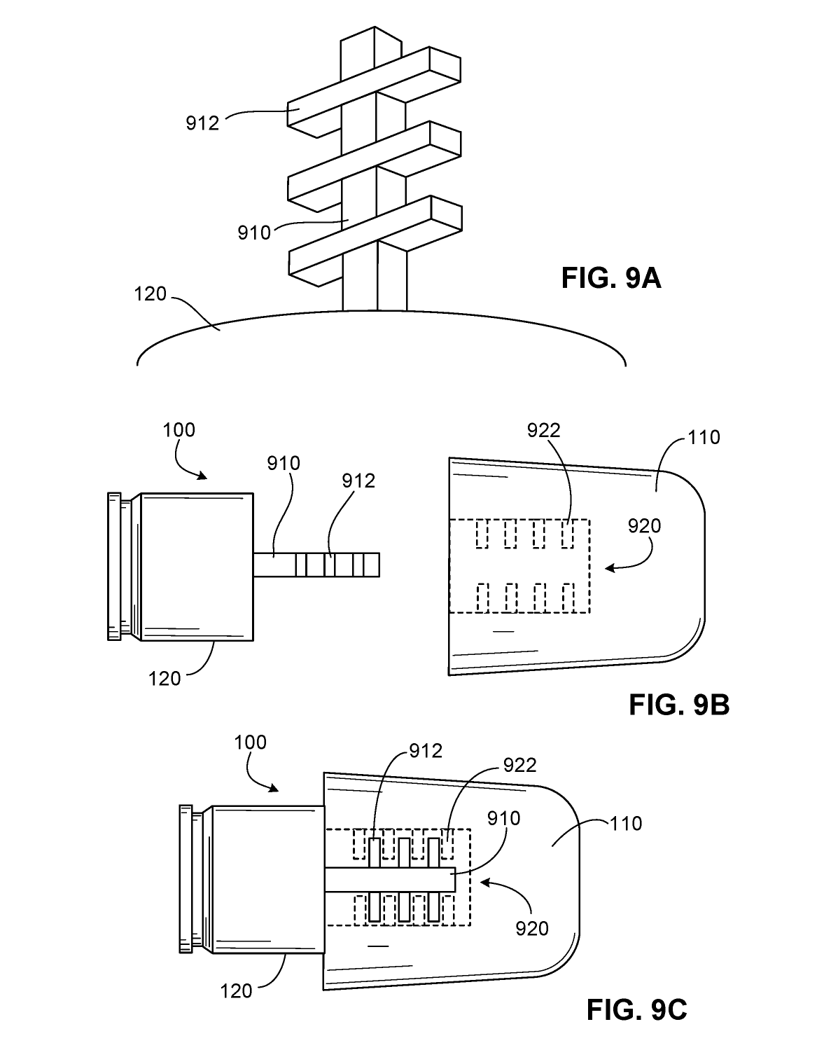

As another example, as shown in FIGS. 9A-C, a plug portion 110 can be coupled to a base portion 120 through an elongate mounting portion 910. The mounting portion 910 is attached to and protrudes from the base portion 120, and includes several teeth 912 extending in opposites directions from the mounting portion 910.

The mounting mechanism 910 can be used to securely and reversibly couple the base portion 120 to the plug portion 110. For example, as shown in FIG. 9B, to mount the base portion 120 to the plug portion 110, a user can rotate the base portion 120 such that the teeth 912 are aligned with a channel 920 defined within the plug portion 110. The user then inserts the mounting portion 910 into the channel 920.

As shown in FIG. 9C, to secure the base portion 120 to the plug portion 110, the user rotates the base portion 120 such that the teeth 912 interlock with teeth 922 protruding from the channel 920. As a result, the teeth 912 are physically locked into place between the teeth 922, thereby coupling the base portion 120 to the plug portion 110.

To separate the base portion 120 from the plug portion 110, the user can rotate the base portion 120 with respect to the plug portion 110 such that the teeth 912 and 922 are no longer interlocked, and pull the base portion 120 away from the plug portion 110.

Although six teeth 912 are shown in FIGS. 9A-C, this is merely an illustrative example. In practice, the mounting portion 910 can include any number of teeth (e.g., one, two, three, four, or more). Similarly, although eight teeth 922 are shown in FIGS. 9B and 9C, this is also merely an illustrative example. In practice, the channel 920 can include any number of teeth (e.g., one, two, three, four, or more).

Further, in FIGS. 9A-C, the teeth 912 are depicted as extending in opposite directions from the mounting portion 910. As a result, the base portion 120 can be rotated approximately 90° relative to the plug portion to secure the base portion 120 to the plug portion 110 (and correspondingly, rotated another approximately 90° to separate the two). In practice, however, the teeth 912 and can be distributed in different patterns, such that the user can rotate the base portion 120 a greater amount (e.g., more than 90°) or a lesser amount (e.g., less than 90°) to secure and/or release the base portion 120 from the plug portion 110. For example, in some cases, the user can rotate the base portion 120 by approximately 45° to secure and/or release the base portion 120 from the plug portion 110.

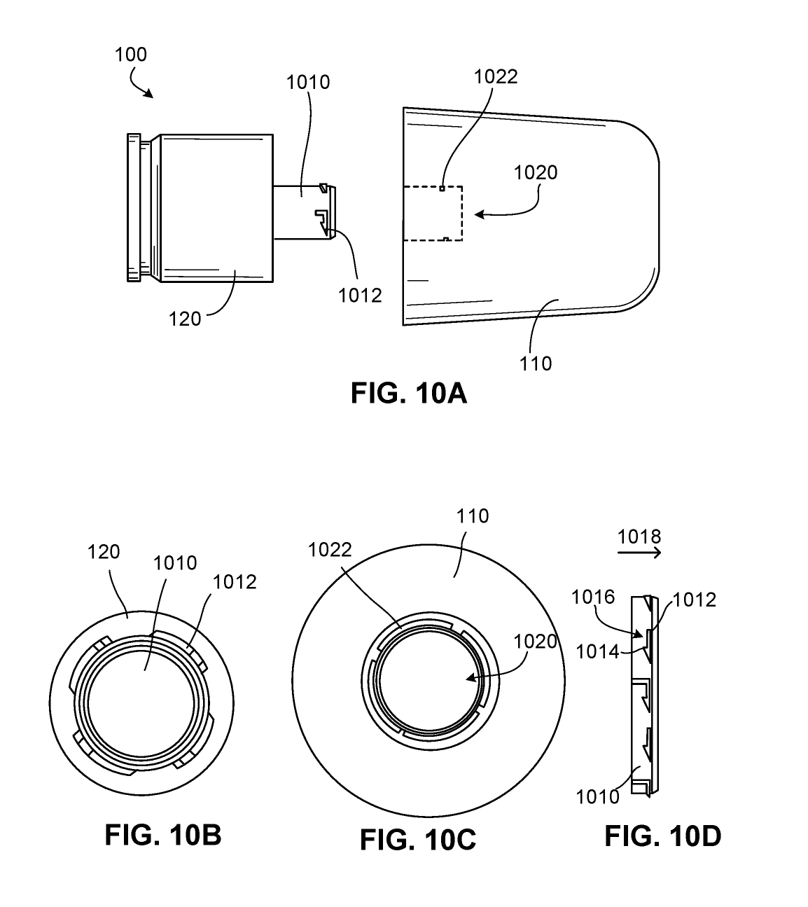

As another example, as shown in FIGS. 10A-D, a plug portion 110 can be coupled to a base portion 120 through an elongate mounting portion 1010. The mounting portion 1010 is generally similar to the mounting portion 910 shown in FIGS. 9A-C. For example, the mounting portion 1010 is attached to and protrudes from the base portion 120, and includes several teeth 1012 extending from the mounting portion 1010.

In this example, however, the teeth 1012 extend in four different directions from the mounting portion 1010, and are equally distributed about the periphery of the mounting portion 1010. This is shown, for example, in FIG. 10B, which depicts a longitudinal view (e.g., head on view) of the mounting portion 1010.

The mounting mechanism 1010 can be used to securely and reversibly couple the base portion 120 to the plug portion 110. For example, to mount the base portion 120 to the plug portion 110, a user can rotate the base portion 120 such that the teeth 1012 are aligned with a channel 1020 defined within the plug portion 110. The user then inserts the mounting portion 1010 into the channel 1020. The channel 1020 is shown in greater detail in FIG. 10C, which depicts a longitudinal view (e.g., head on view) of the channel 1020.

To secure the base portion 120 to the plug portion 110, the user rotates the base portion 120 such that the teeth 1012 couple with teeth 1022 protruding from the channel 1020. As a result, the teeth 1012 are physically locked into place by the teeth 1022, thereby coupling the base portion 120 to the plug portion 110.

In some cases, the teeth 1012 can include structural elements that rotationally lock the base portion 120 to the plug portion 110 when the base portion 120 and the plug portion 110 are coupled together. To unlock the base portion 120 from the plug portion 110, a user can apply a longitudinal force (e.g., by longitudinally displacing the base portion 120 further into the plug portion 110) as he is applying a rotational force to the base portion 120. This can be useful, for example, as it further secures the base portion 120 to the plug portion 110, and reduces or eliminates accidental decoupling when the base portion 120 and/or plug portion 110 are touched by the user.

This functionality can be provided through longitudinal protrusions that extend from each of the teeth 1012. For example, as shown in FIG. 10D (showing a flattened view of the mounting portion 1010), the mounting portion 1010 can include teeth 1012, each of which includes a longitudinal protrusion 1014. When the teeth 1012 are locked with the teeth 1022 of the channel 1020, the teeth 1022 rest within recesses 1016 formed by the longitudinal protrusions 1014 and the teeth 1012. To release the teeth 1022 from the recesses 1016, the teeth 1012 and longitudinal protrusions 1014 must be shifted longitudinally (e.g., in the direction of arrow 1018), such that the teeth 1022 clear the end of the longitudinal protrusions 1014. Thus, when the base portion 120 is locked to the plug portion 110, the two cannot freely rotate with respect to one another until a longitudinal force is also applied.

Although four teeth 1012 are shown in FIGS. 10A-D, this is merely an illustrative example. In practice, the mounting portion 1010 can include any number of teeth (e.g., one, two, three, four, or more).

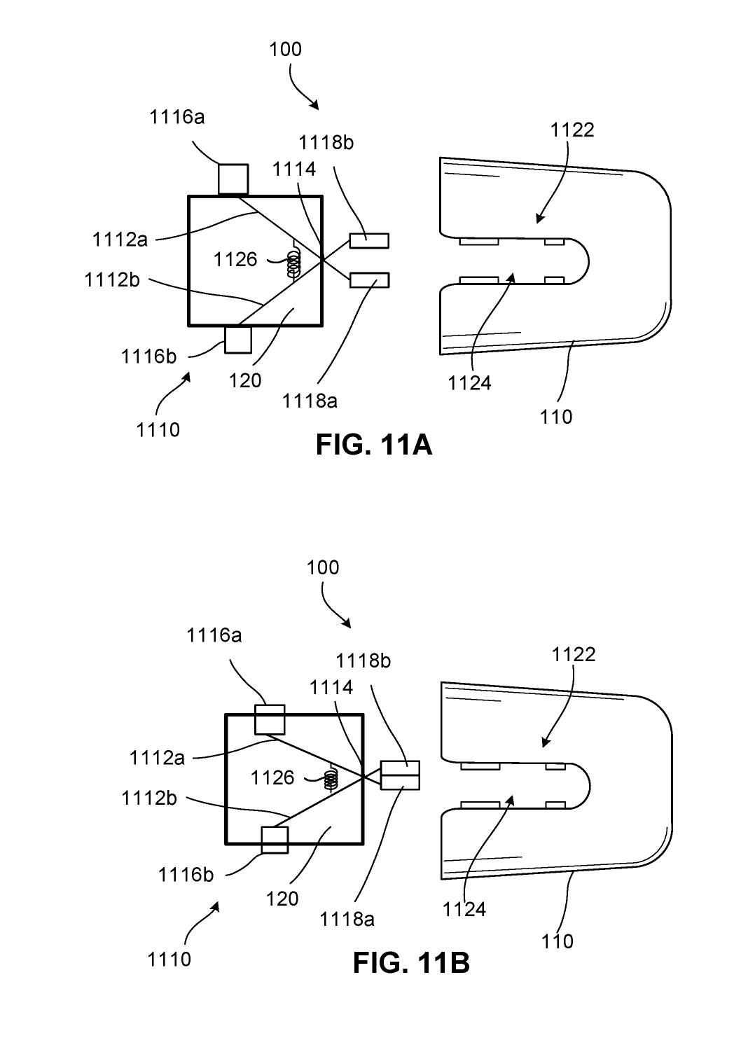

As another example, as shown in FIGS. 11A and 11B, a plug portion 110 can be coupled to a base portion 120 through a mounting mechanism 1110 disposed within the base portion 120.

As shown in FIG. 11A, the mounting mechanism 1110 includes two arms 1112a and 1112b coupled via a hinge 1114 and a spring 1126. The arm 1112a includes a protrusion 1116a proximate an end of the arm 1112a, and the arm 1112b includes a protrusion 1116b proximate an end of the arm 1112b. The protrusions 1116a and 1116b extend from a periphery of the base portion 120 (e.g., protrude out from the base portion 120 as buttons). The arm 1112a also includes a support element 1118a positioned proximate to an end of the arm opposite the protrusion 1114a. Likewise, the arm 1112b includes a support element 1118b positioned proximate to an end of the arm opposite the protrusion 1114b.

The mounting mechanism 1110 can be used to securely and reversibly couple the base portion 120 to the plug portion 110. For example, as shown in FIG. 11B, to mount the base portion 120 to the plug portion 110, a user can press the protrusions 1116a and 1116b into or towards the base portion 120. This force causes the arms 1112a and 1112b to move with respect to the hinge. As a result, the support elements 1118a and 1118b are moved towards each other and closer together, and can be inserted into channel 1122 defined within the plug portion 110.

The user inserts the mounting portion support elements 1118a and 1118b in the channel 1120, and releases the protrusions 1116a and 1116b. The spring 1126 biases the arms 1112a and 1112b back towards their original positions. As a result, the support elements 1118a and 1118b move away from each, and thus, are radially biased against the channel 1120 and rest within recesses 1124 defined within the channel 1122. Thus, the base portion 120 is securely coupled to the plug portion 110.

To release the base portion 120 from the plug portion 110, the user again presses the protrusions 1116a and 1116b into the base portion 120. This force causes the arms 1112a and 1112b to again move with respect to the hinge. As a result, the support elements 1118a and 1118b towards each other. Thus, the support elements 1118 and 1118b are radially biased away from the channel and retract from the recesses 1124. Thus, the plug portion 110 and the base portion 120 can be physically separated from one other by pulling the plug portion 110 and the base portion 120 away from one another.

Although two support elements 1118a and 1118b are shown in FIGS. 11A and 11B, this is merely an illustrative example. In practice, the mounting mechanism 1110 can include any number of support elements (e.g., one, two, three, four, or more). For example, in some cases, the mounting mechanism 1110 can include two longitudinally spaced support elements coupled to the arm 1112a and two longitudinally spaced support elements coupled to the arm 1112b. Correspondingly, the channel 1122 1220 can define multiple longitudinally spaced recesses (e.g., along the length of the channel 1122) to receive the support elements.

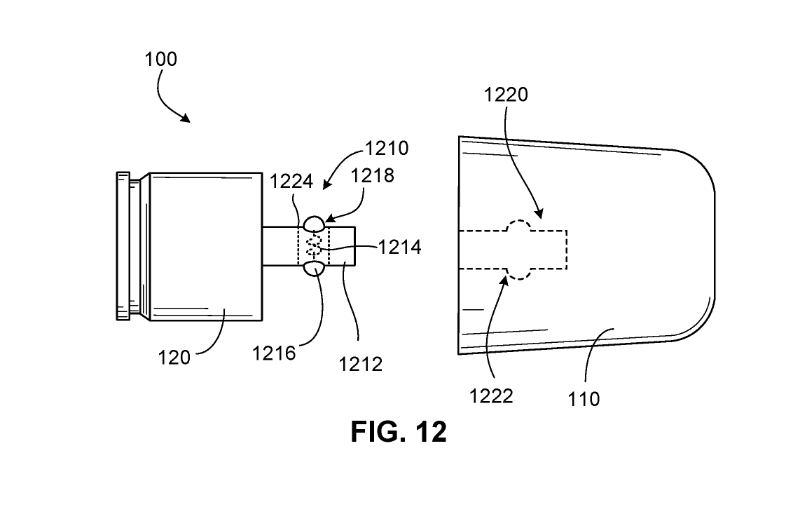

As another example, as shown in FIG. 12, a plug portion 110 can be coupled to a base portion 120 through a mounting mechanism 1210.

The mounting mechanism 1210 is coupled to the base portion 120, and includes an outer sleeve 1212, a spring 1214 disposed within the outer sleeve 1212, and support elements 1216 that protrude from apertures 1218 of the outer sleeve 1212. The spring 1214 is enclosed by an inner sleeve 1224 positioned within the outer sleeve 1212.

The mounting mechanism 1210 can be used to securely and reversibly couple the base portion 120 to the plug portion 110. For example, to mount the base portion 120 to the plug portion 110, a user can insert the outer sleeve 1212 into a corresponding channel 1220 defined within the plug portion 110. As the outer sleeve 1212 enters the channel 1220, the support elements 1216 are forced towards each other and against the bias of the spring 1214 by the walls of the channel 1220. As a result, the support elements 1216 retract into the apertures 1216 (e.g., into the inner sleeve 1224).

Once the outer sleeve 1212 is inserted sufficiently far into the channel 1220, the support elements 1216 become aligned with recesses 1222 in the channel 1220. Due to bias of the spring 1214, the support elements 1216 protrude from the apertures 1218 and into the recesses 1222. Thus, the protruding support elements 1216 secure the mounting mechanism 1210 in place within the channel 1220, thereby coupling the base portion 120 to the plug portion 110.

To release the base portion 120 from the plug portion 110, a user can pull the base portion 120 away from the plug portion 110. As the outer sleeve 1212 exits the channel 1220, the support elements 1216 are again forced towards each other and against the bias of the spring 1214 by the walls of the channel 1220, and retract into the apertures 1218 (e.g., into the inner sleeve 1224). Thus, the base portion 120 can be detached from the plug portion 110.

Although two support elements 1216 are shown in FIG. 12, this is merely an illustrative example. In practice, the mounting mechanism 1210 can include any number of support elements (e.g., one, two, three, four, or more). Further, although a single spring 1214 is shown, in practice, there may be any number of springs (e.g., one, two three, four, or more) positioned either with a single support element alone or between multiple different support elements.

Although several different mechanisms for securing a base portion 120 to a plug portion 110 are described, these are merely illustrate examples. In practice, other mechanisms can also be used, either in addition to or instead of those described herein.

Further, although example hearing protection devices as illustrated in the figures above, these are merely illustrative examples, and may not be drawn to scale. In some cases, certain portions of the hearing protection devices may be enlarged or exaggerated to better depict certain structural features. Accordingly, implementations of the hearing protection devices can differ in size and shape, depending in on the implementation.

A number of embodiments have been described. Nevertheless, it will be understood that various modifications may be made without departing from the spirit and scope of the disclosure. Accordingly, other embodiments are within the scope of the following claims.

1. The hearing protection device of claim 47, wherein the variations in thickness combine to form at least a first protrusion between the first and second ends of the mounting portion;
wherein the mounting portion:
smoothly increases in annular thickness from first point between the first end and the second end to a second point between the first point and the second end, and
smoothly decreases in annular thickness from the second point to the second end.

2. The hearing protection device of claim 1, wherein the mounting portion:
monotonically decreases in thickness from the first end to the first point, monotonically increases in annular thickness from the first point to the second point, and monotonically decreases in annular thickness from the second point to the second end.

3. The hearing protection device of claim 1, wherein the channel is generally tube-like in shape.

4. The hearing protection device of claim 1, wherein an inner diameter of the channel varies between a first end of the channel and a second end of the channel.

5. The hearing protection device of claim 1, wherein the channel is defined by an elastic material.

6. The hearing protection device of claim 1, wherein the channel extends across a full length of the plug portion.

7. The hearing protection device of claim 1, wherein the mounting portion is substantially continuous between the first end and the second end.

8. The hearing protection device of claim 1, wherein the plug portion comprises a compressible material.

9. The hearing protection device of claim 1, wherein the base portion comprises a decorative portion that extends outside of the user's ear when the plug portion is inserted into the user's ear canal.

10. The hearing protection device of claim 1, wherein the plug portion is configured to attenuate sound entering into the user's ear when the plug portion is at least partially inserted into the user's ear canal.

11. A hearing protection device, comprising:
a base portion;
an elongate mounting portion protruding from the base portion, wherein the mounting portion comprises a first end fixed to the base portion, a second end opposite the first end, and a protrusion between the first and second ends;
wherein the protrusion smoothly increases in annular thickness from the annular thickness of the first end of the mounting portion to a maximum annular thickness and smoothly decreases in annular thickness from the maximum annular thickness toward the second end of the mounting portion; and
a plug portion detachably secured to the mounting portion, wherein the plug portion is configured to at least partially insert into a user's ear canal.

12. The hearing protection device of claim 11, wherein the plug portion defines a channel, and the mounting portion is configured to insert into the channel to secure the mounting portion of the plug portion.

13. The hearing protection device of claim 12, wherein the channel defines a recess for receiving the protrusion on the mounting portion.

14. A hearing protection device, comprising:
a base portion;
an elongate mounting portion protruding from the base portion, wherein the mounting portion comprises:
a first end fixed to the base portion,
a second end opposite the first end, and
a first annular protrusion and a second annular protrusion disposed between first end and the second end;
wherein the first protrusion smoothly increases in annular thickness from the annular thickness of the first end of the mounting portion to a maximum annular thickness and smoothly decreases in annular thickness from the maximum annular thickness toward the second protrusion;
wherein the second protrusion smoothly increases in annular thickness from the annular thickness of the mounting portion between the first protrusion and the second protrusion to a maximum annular thickness and smoothly decreases in annular thickness from the maximum annular thickness toward the second end of the mounting portion; and
a plug portion detachably secured to the mounting portion and configured to at least partially insert into a user's ear canal, wherein the plug portion defines a channel having a first recess configured to accept the first annular protrusion and a second recess configured to accept the second annular protrusion.

15. The hearing protection device of claim 14, wherein the plug portion comprises a flexible material, and wherein the channel is defined by the flexible material.

16. The hearing protection device of claim 15, wherein the flexible material comprises a rubber.

17. The hearing protection device of claim 14, wherein the plug portion comprises a compressible material, and wherein the compressible material is disposed along an outer periphery of the compressible material.

18. The hearing protection device of claim 17, wherein the compressible material comprises a foam.

19. A hearing protection device, comprising:
a base portion defining a channel;
a clamp mechanism having a plurality of clamp elements, wherein the clamp element is at least partially disposed within the channel and wherein the clamp element is configured to shift longitudinally within the channel between an open position and a closed position;
a plug portion detachably secured to the base portion via the clamp mechanism and configured to at least partially insert into a user's ear canal,
wherein in the closed position, the plurality of clamp elements grasp a periphery of the plug portion to secure the plug portion to the base portion, and
wherein in the open position, the plurality of clamp elements release the plug portion from the base portion.

20. The hearing protection device of claim 19, wherein the clamp portion comprises at least two clamp elements.

21. The hearing protection device of claim 19, wherein the clamp portion further comprises:
a button; and
a spring disposed between the clamp elements and the button;
wherein when the button is depressed, the spring shifts the clamp portion to the open position; and
wherein when the button is released, the spring shifts the clamp portion to the closed position.

22. A hearing protection device, comprising:
a base portion defining a first channel;
a mounting portion at least partially disposed within the first channel and configured to longitudinally shift within the first channel between a first position and a second position, wherein the mounting portion comprises:
an elongate shaft member; and
a plurality of support elements, the support elements having a smoothly varying perimeter; and
a plug portion detachably secured to the base portion via the support elements and configured to at least partially insert into a user's ear canal, the plug portion defining a second channel,
wherein in the first position, the shaft member radially biases the support elements against an interior of the second channel of the plug portion to secure the plug portion to the base portion, and
wherein in the second position, the shaft member radially biases the support elements away from the second channel of the plug portion to release the plug portion from the base portion.

23. The hearing protection device of claim 22, wherein the shaft member comprises a groove configured to receive the support elements;
wherein in the first position, the support elements are positioned within the groove; and
wherein in the second position, the support elements are positioned outside of the groove.

24. The hearing protection device of claim 23, wherein the groove is defined along an outer circumference of the shaft member.

25. The hearing protection device of claim 22, wherein the mounting portion further comprises:
a button; and
a spring disposed about a periphery of the elongate shaft member;
wherein when the button is depressed, the mounting portion shifts to the open position; and
wherein when the button is released, the mounting portion shifts to the closed position.

26. A hearing protection device, comprising:
a base portion, wherein the base portion defines a first channel;
a mounting portion at least partially disposed within the first channel and configured to rotate within the channel between a first rotational position and a second rotational position, wherein the mounting portion comprises:
a gear member; and
a plurality of support elements, and
a plug portion detachably secured to the base portion via the support elements and configured to at least partially insert into a user's ear canal, wherein the plug portion defines a second channel,
wherein in the first rotational position, the gear member radially biases the support elements against an interior of the second channel of the plug portion to secure the plug portion to the base portion, and
wherein in the second rotational position, the shaft member radially biases the support elements away from an interior of the second channel of the plug portion to release the plug portion from the base portion.

27. The hearing protection device of claim 26, wherein the gear member comprises a plurality of grooves each configured to receive one of the support elements;
wherein in the first rotational position, the support elements are positioned within a respective groove; and
wherein in the second rotational position, the support elements are positioned outside of a respective groove.

28. The hearing protection device of claim 27, wherein the grooves are angularly spaced about a circumference of the gear member.

29. The hearing protection device of claim 26, wherein the first rotational position and the second rotational position differ by approximately 30°.

30. A hearing protection device, comprising:
a base portion comprising a first magnet,
a plug portion comprising a second magnet, wherein the plug portion is detachably secured to the mounting portion via a magnetic force between the first magnetic and the second magnet, and wherein the plug portion is configured to at least partially insert into a user's ear canal.

31. The hearing protection device of claim 30, wherein the first magnet is disposed within a first recess defined by the base portion, and wherein the second magnet is disposed within a second recess defined by the plug portion.

32. A hearing protection device, comprising:
a base portion,
an elongate mounting portion protruding longitudinally from the base portion, wherein the elongate mounting portion comprises a plurality of first teeth protruding radially from the elongate mounting portion;
a plug portion defining a longitudinally extending channel, wherein the plug portion comprises a plurality of second teeth protruding radially into the channel, and wherein the plug portion is detachably secured to the base portion via the first teeth and second teeth, and wherein the plug portion is configured to at least partially insert into a user's ear canal;
wherein when the elongate mounting portion is in a first rotational position relative to the base portion, the first teeth engage with the second teeth to secure the plug portion to the base portion; and
wherein when the elongate mounting portion is in a second rotational position relative to the base portion, the first teeth disengage from the second teeth to release the plug portion from the base portion.

33. The hearing protection device of claim 32, wherein one or more of the first teeth protrude radially from the elongate mounting portion in a first direction, and wherein one or more of the first teeth protrude radially from the elongate mounting portion in a second direction opposite the first direction.

34. The hearing protection device of claim 32, wherein one or more of the second teeth protrude radially into the channel in a first direction, and wherein one or more of the second teeth protrude radially into the channel in a second direction opposite the first direction.

35. The hearing protection device of claim 32, wherein the first rotational position and the second rotational position differ by approximately 90°.

36. The hearing protection device of claim 32, wherein the first teeth are angularly spaced about a circumference of the elongate mounting portion.

37. The hearing protection device of claim 32, wherein the second teeth are angularly spaced about a circumference of the channel.

38. The hearing protection device of claim 32, wherein the first rotational position and the second rotational position differ by approximately 45°.

39. The hearing protection device of claim 32, wherein the first teeth and the second teeth are configured such that when the elongate mounting portion is in a first longitudinal position relative to the base portion, the elongate mounting portion and the base portion are rotationally locked, and
when the elongate mounting portion is in a second longitudinal position relative to the base portion, elongate mounting portion and base portion are not rotationally locked.

40. A hearing protection device, comprising:
a base portion;
a mounting portion comprising a first arm and a second arm, and configured to form first and second positions,
wherein the first arm comprises a first protrusion proximate to an end of the first arm, and a first support element proximate to another end of the first arm, wherein the second arm comprises a second protrusion proximate to an end of the second arm, and a second support element proximate to another end of the second arm, and
wherein the first arm and the second arm are coupled to each other via a hinge, and wherein the first arm and the second arm are configured to move with respect to each other via the hinge;
a plug portion detachably secured to the base portion via the support elements and configured to at least partially insert into a user's ear canal, the plug portion defining a channel;
wherein in the first position, the mounting portion biases the first support element and the second support element against an interior of the channel of the plug portion to secure the plug portion to the base portion, and
wherein in the second position, the mounting portion biases the first support element and the second support element away from the interior of the channel of the plug portion to release the plug portion to the base portion.

41. The hearing protection device of claim 40, wherein the first protrusion and the second protrusion protrude from the base portion;
wherein when the first protrusion and the second protrusion are depressed, the mounting portion is placed into the first position; and
wherein when the first protrusion and the second protrusion are released, the mounting portion is placed into the second position.

42. The hearing protection device of claim 40, wherein the plug portion further defines an annular groove within the channel,
wherein in the first position, the mounting portion shifts the first support element and the second support element into the groove; and
wherein in the second position, the mounting portion shifts the first support element and the second support element out of the groove.

43. The hearing protection device of claim 40, wherein the mounting portion further comprises a spring coupled to the first arm and the second arm,
wherein when the first protrusion and the second protrusion are released, the mounting portion is placed into the second position via a spring force against the first arm and the second arm.

44. A hearing protection device, comprising:
a base portion;
an elongate mounting portion protruding from the base portion, wherein the elongate mounting portion comprises:
an outer sleeve defining a plurality of apertures;
a plurality of moveable support elements protruding from the apertures, and
a spring element disposed between the support elements; and
a plug portion detachably secured to the base portion via the support elements and configured to at least partially insert into a user's ear canal, wherein the plug portion defines a channel,
wherein the elongate mounting portion is configured such that when the elongate mounting portion is inserted a first distance into the channel, the support elements retract into the apertures, and when the elongate mounting portion is inserted a second distance into the channel greater than the first distance, the support elements protrude from the apertures into a recess defined within the channel.

45. The hearing protection device of claim 44, wherein the elongate mounting portion is configured such that when the elongate mounting portion is withdrawn from within the channel by a third distance, the support elements retract into the apertures, and when the elongate mounting portion is withdrawn from within the channel by a fourth distance greater than the third distance, the support elements protrude from the apertures.

46. The hearing protection device of claim 44, wherein the elongate mounting portion further comprises an inner sleeve disposed within the outer sleeve and surrounding the spring element.

47. A hearing protection device, comprising:
a base portion;
an elongate mounting portion protruding from the base portion,
wherein the mounting portion comprises a first end fixed to the base portion and a second end opposite the first end, and
wherein the mounting portion includes variations in thickness along a length between the first and second ends, and
a plug portion detachably secured to the mounting portion, wherein the plug portion defines a channel, and the mounting portion is configured to insert into the channel to secure the mounting portion to the plug portion, wherein the plug portion is configured to at least partially insert into a user's ear canal.

48. The hearing protection device of claim 47, wherein:
the variations combine to form a plurality of annular recesses.

49. The hearing protection device of claim 48, wherein:
the mounting portion initially decreases in thickness from a first point between the first end and the second end to a second point between the first point and the second end; and
the mounting portion increases in thickness from a third point between the second point and the second end to a fourth point between the third point and the second end, forming a first of the plurality of annular recesses between the second and fourth points.

50. The hearing protection device of claim 49, wherein:
the mounting portion decreases in thickness from a fifth point between the fourth point and the second end; and
the mounting portion increases in thickness from a sixth point between the fifth point and the second end to a seventh point between the sixth point and the second end, forming a second of the plurality of annular recesses between the fifth and seventh points.

51. The hearing protection device of claim 48, wherein the annular recesses have walls perpendicular to a longitudinal axis of the mounting portion.

52. The hearing protection device of claim 48, wherein the annular recesses have walls angled away from perpendicular to a longitudinal axis of the mounting portion.

53. The hearing protection device of claim 48, wherein the plurality of annular recesses comprises two or more annular recesses.

54. The hearing protection device of claim 48, wherein the channel includes an annular ridge along a wall of the channel.

55. The hearing protection device of claim 55, wherein the annular ridge aligns with one of the plurality of annular recesses in the mounting portion.

56. The hearing protection device of claim 47, wherein the channel has a generally circular cross-section or a cross-sectional shape similar to a shape of the mounting portion.

57. The hearing protection device of claim 47, wherein a diameter of the channel is smaller than a diameter of the mounting portion.

58. The hearing protection device of claim 48, wherein the plug portion comprises a compressible material.

59. The hearing protection device of claim 48, wherein the channel is defined by an elastic material.
